(19) United States
(12) Patent Application Publication
(10) Pub. No.: US 2003/0232427 A1
(43) Pub. Date: Dec. 18, 2003

(54) Optically active substrates for examination of biological materials

(76) Inventor: Jean I. Montagu, Brookline, MA (US)

Correspondence Address: Chief IP Counsel, Legal Department, AFFYMETRIX, INC., 3380 Central Expressway, Santa Clara, CA, 95051, US

(21) Appl. No.: 10/462,944

(22) Filed: Jun. 17, 2003

Related U.S. Application Data
(60) Provisional application No. 60/389,554, filed on Jun. 18, 2002.
(60) Provisional application No. 60/463,392, filed on Apr. 16, 2003.

(51) Int. Cl. C12M 1/34; G01J 3/00

(52) U.S. Cl. 435/287.2; 356/300

(57) ABSTRACT

An optical apparatus for examination of biological material, a light source, a light detector and several optical elements for providing light from the light source to an optical substrate. The optical substrate having a surface comprising a dense array of micro-optical elements in close proximity to biological material being examined. The optical apparatus may include optical elements such as a lens that is arranged to provide light to and collect light from the dense array of micro-optical elements and the biological material. The lens and the dense array of micro-optical elements provide optical properties that increase the number of photons interacting with the biological material or increase the number of photons collected by the lens after interaction with the biological material.

This application claims priority from U.S. Provisional Application 60/389,554, filed on Jun. 18, 2002 and U.S. Provisional Application 60/463,392, filed on Apr. 16, 2003, which are incorporated by reference.

### BACKGROUND OF THE INVENTION

The present invention relates to optically examining, detecting, or analyzing biological materials, and particularly relates to examining, detecting or imaging biological polymers located on or near optically active substrates.

Microarray technology enables studying complex biochemical reactions and systems at once instead of studying them individually. The technology provides a massively parallel form of analysis that increases data collection per unit time, decreases the overall time required for analysis, uses smaller sample volumes and reagent volumes and sometimes reduces disposable consumption. Although the initial cost may be high, overall the technology represents considerable savings in the time and costs of associated labor. Microarray technology became a fundamental tool for genomic research. The technology can also be utilized for routine analysis used in clinical diagnostics or for industrial analytical purposes.

Microarrays with extremely large number of features are manufactured by methods described in PCT Application WO 92/10092 or U.S. Pat. Nos. 5,143,854; 5,384,261; 5,405,783; 5,412,087; 5,424,186; 5,445,934; 5,744,305; 5,800,992; 6,040,138; 6,040,193 all of which are incorporated by reference. The array features usually have dimensions from about 10 microns to one hundred microns. Each feature can include several million DNA molecules. The synthesis area of a wafer may be about 110 mm×110 mm and may include several individual microarray chips. To examine a microarray, an optical scanner, for example, may need to scan approximately 65,536×65,536 pixels, for microarrays having feature sizes of 10 μm to 12.5 μm, and approximately 32,768×32,768 pixels, for microarrays having feature sizes of 20 μm to 25 μm. Alternatively, microarrays can be fabricated by other techniques as described in PCT Application PCT/US99/18438 published as WO 00/09757, which is incorporated by reference.

Microarrays may also be created by microfluidic delivery, as described in PCT Application PCT/US99/00730, published as WO 99/36760, which is incorporated by reference. These microarrays can contain a wide range of biological materials including, plant, animal, human, fungal and bacteria cells; viruses, peptides, antibodies, receptors, and other proteins; cDNA clones, DNA probes, oligonucleotides, polymerase chain reactions (PCR) products, and chemicals. These biological materials are delivered in form of an array of spots to various microarray substrates including chemically treated microscope slides, coverslips, various wafer, plastics, membranes, or gels. The number of deposited spots may be in the range of 100 to 50,000 per microarray, and the diameter of an individual spot may be in the range of 50 μm to 1000 μm, and preferably 100 μm to 250 μm. The volume of each deposited spot is in the range of 10 pL to 10 nL, and preferably 50 pL to 500 pL, where it is difficult to precisely deliver and measure the liquid volumes.

In general, fluorescence microscopy is a relatively inefficient process, wherein the light source-to-detector efficiency is estimated in parts per trillions. There is usually a very low efficiency of the fluorescence conversion. Furthermore, among other limitations, the scanning microscope systems cannot increase the intensity of the illumination by the laser source, because the fluorescent sample would be destroyed; this is known as photo-bleaching. Also, before photo-bleaching takes place, most fluorophores behave in a non-linear and possibly unpredictable manner. Additionally, numerous non-optical constrains come into play such as acceptable scan duration, detector performance, and electronic and image manipulation processes. These factors also affect the signal to noise ratio.

Therefore, there is a need for optical examination, detection or imaging systems that are relatively inexpensive and exhibit a high source-to-detector efficiency.

### SUMMARY OF THE INVENTION

The present invention relates to systems and methods for detecting, examining or analyzing biological materials by optical techniques using novel, optically active substrates or surfaces. The present invention also relates to optically active substrates or surfaces capable of accommodating various probes or microarrays for optically detecting, examining or analyzing biological materials.

An optical system, for use with an optically active substrate or surface, may include a light source, a light detector, and an optical light path system. The use of optically active surface improves detection and examination efficiency of different biological materials. The biological materials include biological polymers such as oligonucleotides to which fluorescently labeled DNA or RNA is bound, polypeptides or other polymer arrays, electrophoresis gels, or other biological specimens such as agonists and antiagonists for cell membrane receptor, toxins and venoms, viral epitopes, hormones (e.g. opioid peptides, steroids, etc.), hormones receptors, peptides, enzimes, enzimes substrates, cofactors, drugs lectins, sugars, oliginucleoteds, nucleic acids, oligosaccharides, proteins, antibodies, cell receptors, monoclonal antibodies and antisera active with specific antigenic determinants (such as viruses, cells and other materials), drugs polynucleotides, nucleic acids, peptides, cofactors, lectins, sugars, polysaccharides, cells, celular membranes and organellesas well as other similar materials.

The optical light path system may include an objective lens. The objective lens and the optically active substrate or surface may form an optical system arranged for efficient delivery of photons to, and efficient collection of photons from, the examined biological material. The optical properties of the objective lens and the optically active substrate or surface may be cooperatively designed for a range of wavelength or a specific geometrical arrangement. The optical lens has the numerical aperture, focal length, size and other parameters, which at least some are co-operatively fabricated with the optical properties of an array of micro-optical elements located on the optically active surface. The objective lens delivers photons to the optically active surface, and collects photons after being scattered, reflected or re-emitted from the examined biological material and/or after interaction with the optically active surface.

According to one aspect, an optical apparatus for examination of biological material includes a surface comprising a dense array of micro-optical elements located in close proximity to biological material being examined. The apparatus also includes a light source, a light detector and optical elements for providing light from the light source to the biological material and from the biological material to the light detector. The optical elements may include or be associated with a lens (or a lens system or an “immersed lens-like” structure) arranged to provide light to and collect light from the dense array of micro-optical elements and the biological material.

According to another aspect, an optically active substrate is constructed for use with an optical system for examination of biological materials. The optically active substrate includes an optical substrate having a first surface and a second surface opposite to the first surface; and a dense array of micro-optical elements associated with the optical substrate and located in close proximity to biological material being examined. The dense array of micro-optical elements is constructed to have optical properties that increase the number of irradiation photons interacting with the biological material or increase the number of photons provided to an external optical system after interaction of the irradiation photons with the biological material.

According to yet another aspect, an optical method for examination of biological material includes using a surface comprising a dense array of micro-optical elements located in close proximity to biological material being examined. The method also includes emitting light from a light source, and detecting light by a light detector, wherein the light has interacted with optical elements for providing light from the light source to the biological material and from the biological material to the light detector.

Preferred embodiments of the above aspects include one or more of the following:

The micro-optical elements may form an integral part of an optically active substrate or just an optically active surface. The micro-optical elements may have at least one dimension comparable to the wavelength of light emitted from the light source. The micro-optical elements may include micro-structures formed at the substrate's surface or inside the substrate. The micro-structures may include micro-lenses. The micro-lenses may be formed by micro-cavities formed inside the substrate.

The micro-lenses may be formed by micro-cavities having parallel or semi-parallel groves in the form of half cylinders or quarter cylinders. The micro-cavities may be formed inside the substrate by spherical indentations about one radius in depth or indentations having a depth less than one radius. Alternatively, the micro-cavities may be formed inside the substrate by indentations having a hyperbolic shape. The micro-cavities may include a surface covered by a layer of a high index medium transparent at a wavelength of the light. The high index medium may substantially fill the micro-cavities.

The micro-optical elements may be micro-lenses formed by micro-cavities inside the substrate, wherein the micro-cavities have a radius in the range of 0.1 μm to 10 μm. In general, the micro-cavities have a radius less than 100 μm.

The micro-optical elements may include micro-lenses formed by micro-cavities inside a substrate, wherein the diameter and depth of the micro-cavities define the thickness of a high index coating deposited on the surface. The substrate may be transparent to fluorescent light emitted from fluorophores excited at their emission wavelength. The high index coating may have a thickness of about 10 angstrom to 1000 angstrom depending on the material so that a relatively low coefficient of transmission of the material causes acceptable optical losses.

The substrate may be optically transparent and can be made of various materials including polycarbonate, Mylar®), PMMA®, Plexiglas®, or a plastic having an index of refraction about 1.57. Alternatively, the substrate may include functionalized glass or other material and the optically active surface may be fabricated, deposited or attached to the substrate.

Additional aspects of the systems and methods for detecting, examining or analyzing biological materials are described in the related PCT Application PCT/US01/20177 filed on Jun. 25, 2001, which is incorporated by reference.

Additional aspects of the systems and methods for detecting, examining or analyzing biological materials are described below.

### BRIEF DESCRIPTION OF THE DRAWINGS

FIG. 1 is a schematic illustration of a confocal optical scanning and imaging system for examination of biological material.

FIG. 2 is a schematic illustration of a CCD wide field optical imaging system for examination of biological material.

FIG. 2A is a simplified graphical representation of an optical scanning system.

FIG. 2B is a perspective view of a scanning arm portion of the scanner shown in FIG. 2A.

FIG. 2C is a top planar view of the scanning arm shown in FIG. 2B.

FIG. 3 illustrates a cross-sectional view of a reflective, optically active substrate.

FIG. 3A is an enlarged view of biological material located on a surface of the optically active substrate shown in FIG. 3.

FIG. 3B illustrates a cross-sectional view of another embodiment a reflective, optically active substrate.

FIG. 4 illustrates a cross-sectional view of yet another embodiment a reflective, optically active substrate.

FIG. 4A is an enlarged view of a micro-optical element created in the optically active substrate shown in FIG. 4.

FIG. 4B is a top view of the optically active substrate shown in FIG. 4 including semi cylindrical grooves.

FIG. 4C is a top view the optically active substrate shown in FIG. 4 including spherical elements.

### DESCRIPTION OF THE PREFERRED EMBODIMENTS

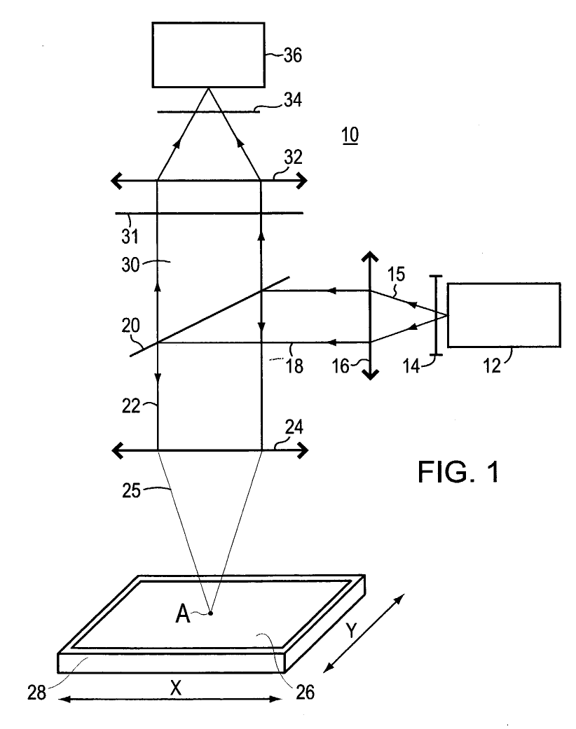

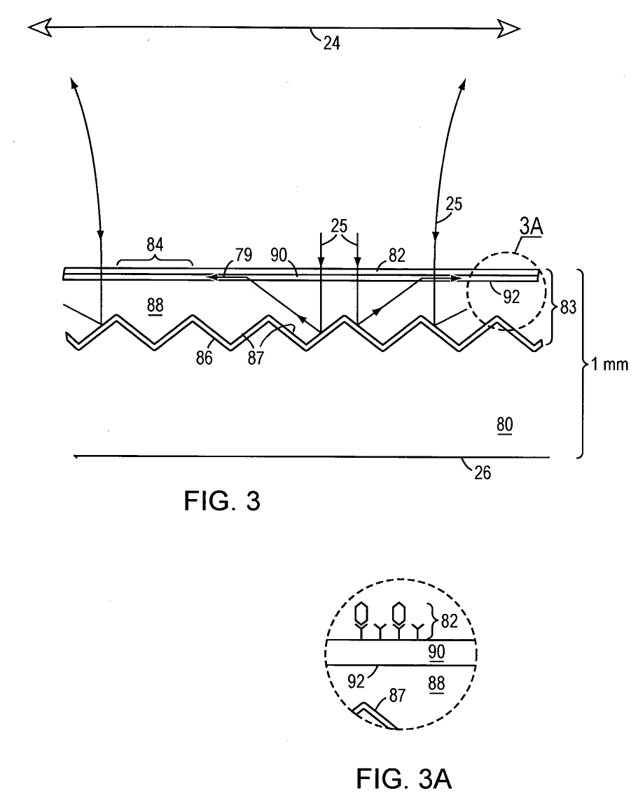

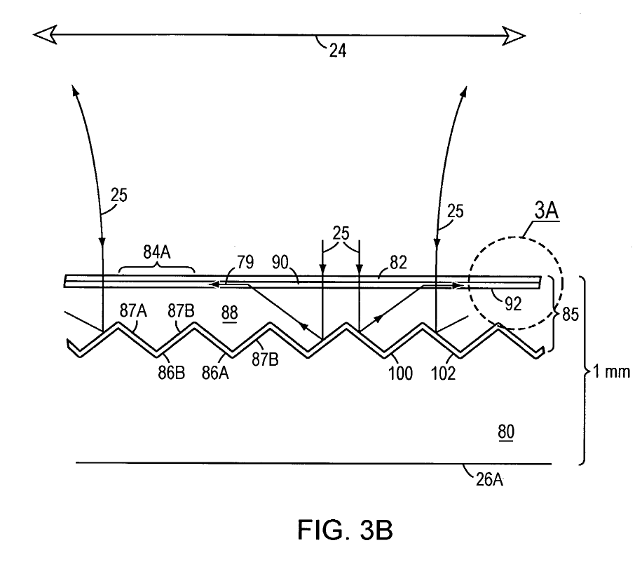

FIG. 1 illustrates one embodiment of a confocal optical scanning and imaging system for examination of biological material located on, or near, an optically active substrate shown in FIG. 3. The optically active substrate forms an important part of the optical system for increasing the detected optical signal.

Referring to FIG. 1, optical system 10 includes a light source 12, an entrance _aperture 14, a lens 16, a dichroic mirror 20, an objective lens 24, a two or three axis translation table 28, a lens 32, an exit pinhole 34, a band pass filter or a rejection filter 31 and a detector 36. In the following embodiment, optical system 10 is arranged for the detection of fluorescent light; however, optical system 10 may also be arranged for the detection of scattered or transmitted light at the irradiation wavelength.

Light source 12 emits an excitation light beam 15, and dichroic mirror 20 directs the excitation light toward objective lens 24. Objective lens 24 focuses light onto a pixel (A) located on or near an optically active substrate 26. The irradiation beam is in the range of about 2 μm to about 10 μm. Fluorescent light emitted from pixel A is collected by objective lens 24 and transmitted through dichroic mirror 20, over a light path 30, toward and trough band pass or rejection filter 31 and to light detector 36. The arrangement of a aperture and pinholes 14 and 34, and lenses 16 and 32 provides fluorescent light from a selected depth (in the Z direction) of pixel A to detector 36, while light emitted from other depths in the Z direction is substantially blocked and doesn't pass through pinhole 34. This spatial filter improves the signal-to-noise ratio. The size of exit pinhole 34 is optimized to improve the resolving power of optical system 10.

Light source 12 is constructed to emit light of a wavelength capable of exciting fluorophores associated with the examined biological tissue. For example, light source 12 includes a gas lasers or diodes lasers or LEDs that emits simultaneously or sequentially _{—}473 or 488 or 490 nm as well as diodes that emit at 532 nm as well as diodes that emit at 638 nm or at 745 light directed to epi-optically active substrate 26 by dichroic mirror 20. For example, the excitation light of 488 nm irradiates a pixel on the surface of substrate 26, and excites fluorophores that emit fluorescent light, for example, in the range 515 nm to 595 nm. Various types of fluorophores (and their corresponding absorption maxima) are Fluorescein (488 nm), Dichloro-fluorescein (525 nm), Hexachloro-fluorescein (529 nm), Tetramethylrhodamine (550 nm), Rhodamine X (575 nm), Cy3™ (550 nm), Cy5™ (650 nm), Cy7™ (750 nm), and IRD40 (785 nm). Detector 36 with suitable band pass or rejection filters, detects the fluorescent light emitted from the pixel of optically active substrate 26. Preferably, objective lens 24 has a relatively large numerical aperture (at least a numerical aperture of 0.1, but preferably, a numerical aperture above 0.5, and more preferably 0.6 or 0.7). Optical system 10 can collect optical data over an array of pixels by displacing epi-optically active substrate 26 in the X and Y directions using a translation table 28.

In general, light source 12 may be a light emitting diode, or a lamp with a filter, but preferably a laser (such as an argon laser, a helium-neon laser, a diode laser, a dye laser, a titanium sapphire laser, a frequency-doubled diode pumped Nd:YAG laser, or a krypton laser). Typically, the excitation source illuminates the sample with an excitation wavelength that is within the visible spectrum, but other wavelengths (i.e., near ultraviolet or near infrared spectrum) may be used depending on the type of markers and/or samples or detection methods. Preferably, the angle of the irradiation is well controlled with respect to the irradiated surface, wherein the alignment provides an aligned beam within the anglular range of a few hundred microradians, and preferably within 100 microradian, in order to launch an evanescent wave as described below. Light detector 36 may be a photomultiplier (PMT), a diode, or another photodetector.

FIG. 3 illustrates one embodiment of an epi-optically active substrate 26 with biological material on a proximal side relative to irradiation beam 25. Epi-optically active substrate 26 includes an optical plate 80 having an optically active surface 83 with a dense array of micro-optical elements 84. The dense array of micro-optical elements 84 has optical properties designed and fabricated to increase the number of photons interacting with the biological material and increase the number of photons collected by lens 24 after interaction with biological material 82. Specifically, micro-optical elements 84 are designed to excite evanescent field used for probing biological material 82 located near the optically active surface, as shown in FIG. 3A. The evanescent field excites fluorescent emission from flourophores, associated with biological material 82, and the fluorescent light is collected by lens 24 and provided to detector 36, as described above.

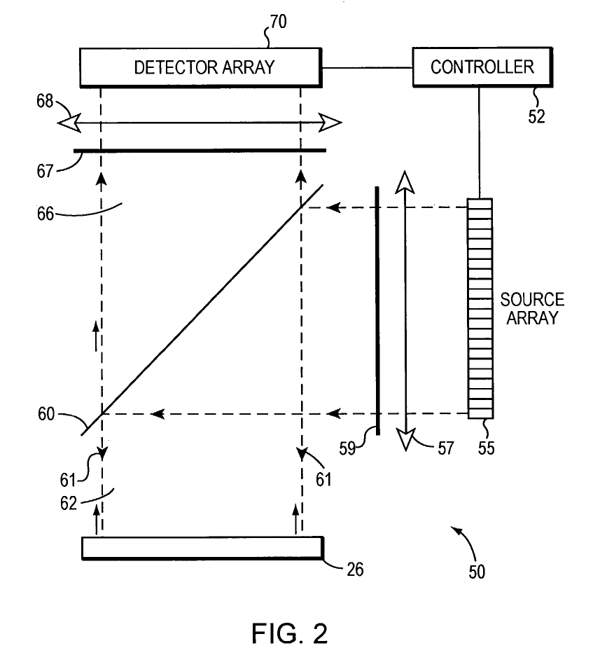

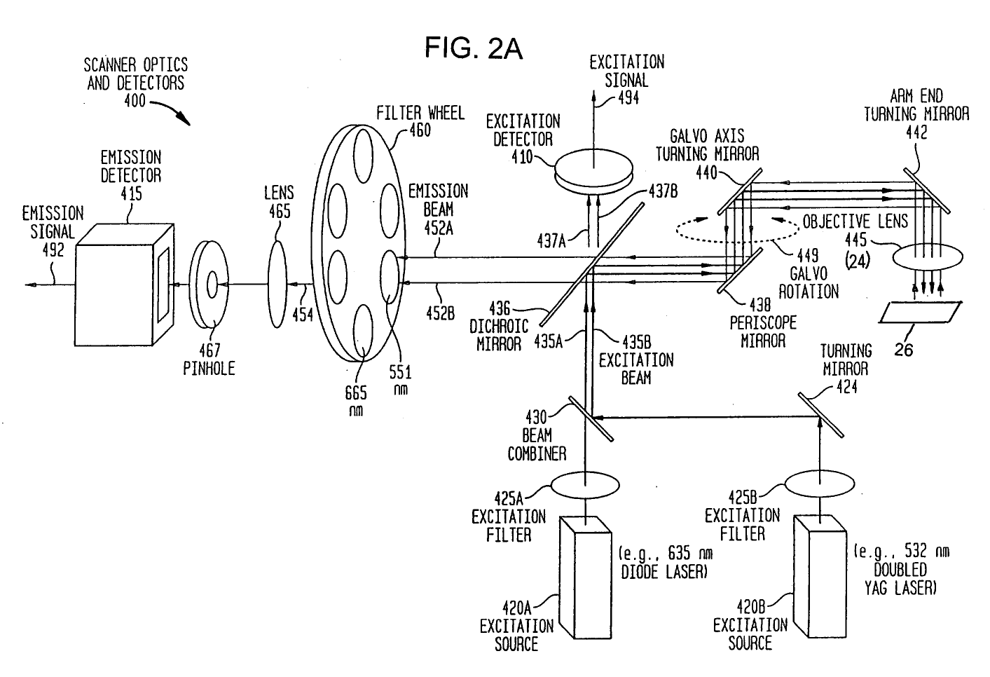

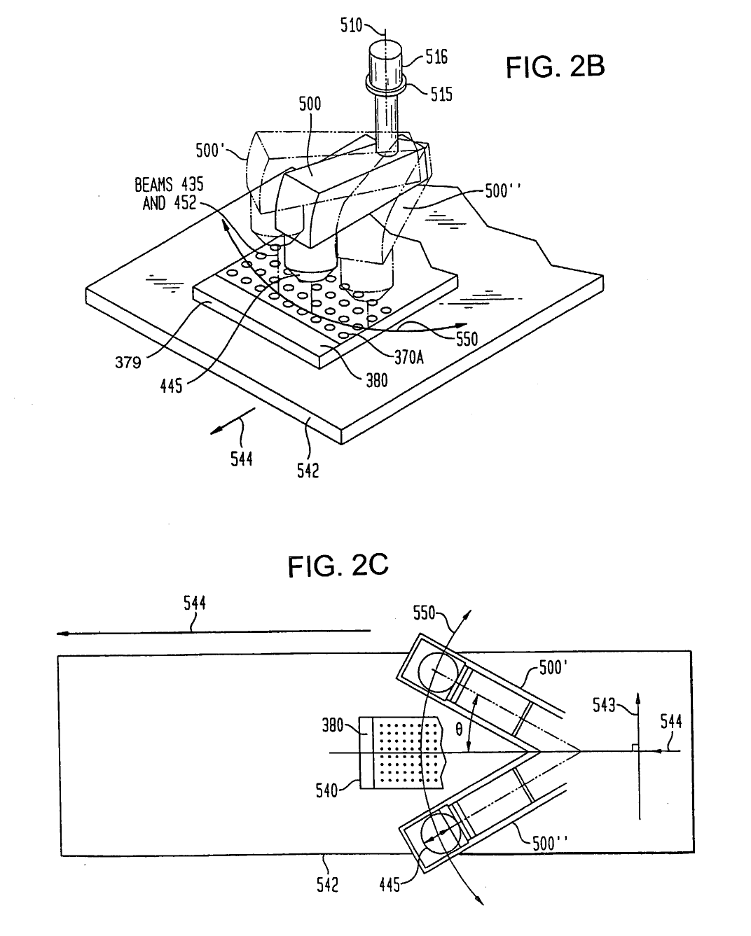

FIG. 2 schematically illustrates another embodiment of an optical imaging system constructed and arranged to examine of biological material located on an optically active substrate. Optical system 50 includes a light source array 55, a lens 57, band pass filter 59, a dichroic mirror 60, a lens 68, band pass or rejection filter 67 and a detector array 70. Light source array 55 emits light directed by lens 57 to filter 59, to dichroic mirror 60, which in turn directs the emitted light beam toward optically active substrate 26, shown in FIG. 3.

Preferably, light source array 55 includes an array of laser diodes or micro-lasers that are aligned to provide a parallel and collimated array of beams. The angle of the beams is controlled within a range of a few hundred microradians and preferably within 100 microradian in order to launch (or excite) evanescent radiation as described below.

Preferably, the optical system is arranged to detect fluorescent light emitted from the examined biological material in response to the evanescent wave. For example, light source array 55 includes a two-dimensional array of elements cooperatively arranged to emit a two-dimensional beam of 490 nm light. This excitation light irradiates a large area of optically active substrate 26 and excites fluorescent light over the irradiated area. Specifically, the two-dimensional beam is directed to dichroic mirror 60, which reflects the light beam to an optical path 62 and enables irradiation of a large area of optically active substrate 26. As described below, the optical elements provide a highly parallel and collimated array of irradiation beams.

A fluorescently labeled biological material, located over the irradiated area, emits fluorescent light, for example, in the range 515 nm to 595 nm. Lens 68 receives, from dichroic mirror 60, fluorescent light emitted from the irradiated area of optically active substrate 26. Rejection filter or band pass filter 67 permits preferably fluorescent radiation to progress toward detector array 70. Detector array 70 detects the fluorescent light over a two-dimensional detection area. The detected signal is digitized and stored individually for each pixel in a memory.

According to another embodiment, the optical system is constructed to probe a one-dimensional area of optically active substrate 26 at a time. Specifically, optical system 50 includes light source array 55 with a one-dimensional array of elements arranged to emit a one-dimensional beam of excitation light (i.e., a strip or ribbon of excitation light) and an excitation band pass filter 57. Optically active substrate may be located on a linear stage and translated under linear light source and detector. The optical system may include a mirror, or other optical elements, arranged to provide the fluorescent light emitted from one line of the examined biological material to a one-dimensional detector array 70. The optical system may optionally include a slit located before the one-dimensional detector array for confocal detection as well as suitable band pass or rejection filters.

In general, light source array 55 may include a one-dimensional or two-dimensional array of micro-lasers, laser diodes, or possibly light emitting diodes (LEDs) wherein each source element may include a separate lens or a similar optical element. Detector array 70 may include an array of photomultiplier tubes (PMTs) or avalanche photodiodes (APDs), or an integrating or non-integrating array of charge coupled devices (CCD). More expensive optical systems 50 may use, as an image detector, a scientific-grade cooled CCD camera or a cooled CCD with an image intensifier, which usually exhibits an excellent geometrical and photometric linearity, a wide dynamic range and good photon detection efficiency.

Optionally, detector array 70 can convert the collected light into video-level electrical signals for display on a video monitor. Optical system 50 may include video output peripheral devices, including a computer frame grabber for digitizing the image information and for storing it in a computer memory.

There are other optical systems suitable for use with epi-optically active substrates 26. These systems may include various light-detectors employing photodiodes, charge-coupled devices, photomultiplier tubes, or similar devices to register the collected emission beams. For example, a scanning system for use with a fluorescently labeled target is described in U.S. Pat. No. 5,143,854, hereby incorporated by reference in its entirety for all purposes. Other embodiments of the scanners or scanning systems are described in U.S. Pat. Nos. 5,578,832, 5,631,734, 5,834,758, 5,936,324, 5,981,956, 6,025,601, 6,141,096, 6,185,030, 6,201,639, 6,218,803, and 6,252,236; in PCT Application PCT/US99/06097 (published as WO99/47964); and in from U.S. patent applications Ser. Nos. 09/682,071; 09/682,074; and 09/682,076 all of which were filed on Jul. 17, 2001, each of the above-listed patent documents is hereby incorporated by reference in their entireties for all purposes.

FIG. 2A is a simplified graphical representation of selected components of an illustrative type of a scanner 400 suitable for scanning hybridized spotted arrays deposited on epi-optically active substrates 26 (i.e., in this example, spotted arrays are after the hybridization process described in PCT Application PCT/US01/26297, which is incorporated by reference). These illustrative components, which will be understood to be non-limiting and not exhaustive, are referred to collectively for convenience as scanner optics and detectors 401. Scanner optics and detectors 401 include excitation sources 420A and 420B (collectively referred to as excitation sources 420). Any number of one or more excitation sources 420 may be used in alternative embodiments. In the present example, sources 420 are lasers; in particular, source 420A is a diode laser producing red laser light having a wavelength of 635 nanometers and, source 420B is a doubled YAG laser producing green laser light having a wavelength of 532 nanometers. Further references herein to sources 420 generally will assume for illustrative purposes that they are lasers, but, as noted, other types of sources, e.g., x-ray sources, may be used in other implementations.

There are other optical systems suitable for use with epi-optically active substrates 26. A typical optical employs optical and other elements to provide an excitation beam, such as from a laser, and to selectively collect the emission beams. These systems may include various light-detectors employing photodiodes, charge-coupled devices, photomultiplier tubes, or similar devices to register the collected emission beams. For example, a scanning system for use with a fluorescently labeled target is described in U.S. Pat. No. 5,143,854, hereby incorporated by reference in its entirety for all purposes. Other scanners or scanning systems are described in U.S. Pat. Nos. 5,578,832, 5,631,734, 5,834,758, 5,936,324, 5,981,956, 6,025,601, 6,141,096, 6,185,030, 6,201,639, 6,218,803, and 6,252,236; in PCT Application PCT/US99/06097 (published as WO99/47964); and in from U.S. patent applications Ser. Nos. 09/682,071; 09/682,074; and 09/682,076 all of which were filed on Jul. 17, 2001, each of these patent documents is hereby incorporated by reference in their entireties for all purposes.

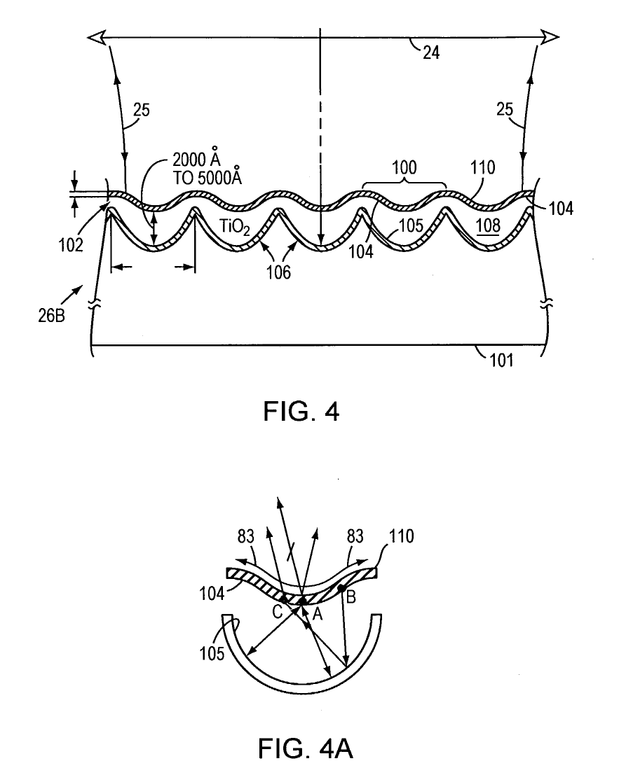

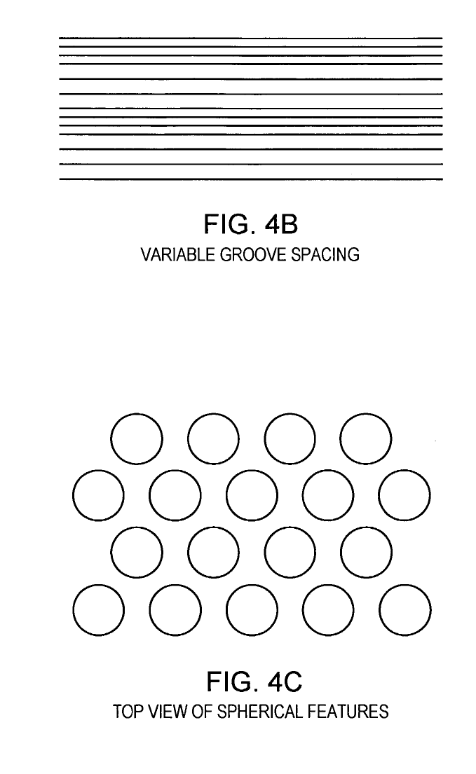

Sources 420A and 420B may alternate in generating their respective excitation beams 435A and 435B between successive scans, groups of successive scans, or between full scans of an array. Alternatively, both of sources 420 may be operational at the same time. For clarity, excitation beams 435A and 435B are shown as distinct from each other in FIG. 4. However, in practice, turning mirror 424 and/or other optical elements (not shown) typically are adjusted to provide that these beams follow the same path.

Scanner optics and detectors 400 also includes excitation filters 425A and 425B that optically filter beams from excitation sources 420A and 420B, respectively. The filtered excitation beams from sources 420A and 420B may be combined in accordance with any of a variety of known techniques. For example, one or more mirrors, such as turning mirror 424, may be used to direct filtered beam from source 420A through beam combiner 430. The filtered beam from source 420B is directed at an angle incident upon beam combiner 430 such that the beams combine in accordance with optical properties techniques well known to those of ordinary skill in the relevant art. Most of combined excitation beams 435 are reflected by dichroic mirror 436 and thence directed to periscope mirror 438 of the illustrative example. However, dichroic mirror 436 has characteristics selected so that portions of beams 435A and 435B, referred to respectively as partial excitation beams 437A and 437B and collectively as beams 437, pass through it so that they may be detected by excitation detector 410, thereby producing excitation signal 494.

In the illustrated example, excitation beams 435 are directed via periscope mirror 438 and arm end turning mirror 442 to an objective lens 445. As shown in FIGS. 5A and 5B, lens 445 in the illustrated implementation is a small, light-weight lens located on the end of an arm that is driven by a galvanometer around an axis perpendicular to the plane represented by galvo rotation 449 shown in FIG. 4. Objective lens 445 thus, in the present example, moves in arcs over hybridized spotted arrays 132 disposed on slide 379. Flourophores in hybridized probe-target pairs of the arrays that have been excited by beams 435 emit emission beams 452 (beam 452A in response to excitation beam 435A, and beam 452B in response to excitation beam 435B) at characteristic wavelengths in accordance with well-known principles. Emission beams 452 in the illustrated example follows the reverse path as described with respect to excitation beams 435 until reaching dichroic mirror 436. In accordance with well-known techniques and principles, the characteristics of mirror 436 are selected so that beams 452 (or portions of them) pass through the mirror rather than being reflected.

In the illustrated implementation, filter wheel 460 is provided to filter out spectral components of emission beams 452 that are outside of the emission band of the fluorophore, thereby providing filtered beams 454. The emission band is determined by the characteristic emission frequencies of those fluorophores that are responsive to the frequencies of excitation beams 435. In accordance with techniques well known to those of ordinary skill in the relevant arts, including that of confocal microscopy, filtered beams 454 may be focused by various optical elements such as lens 465 and also passed through illustrative pinhole 467 or other element to limit the depth of field, and thence impinges upon emission detector 415.

Emission detector 415 may be a silicon detector for providing an electrical signal representative of detected light, or it may be a photodiode, a charge-coupled device, a photomultiplier tube, or any other detection device that is now available or that may be developed in the future for providing a signal indicative of detected light. For convenience of illustration, detector 415 will hereafter be assumed to be a photomultiplier tube (PMT). Detector 415 thus generates emission signal 492 that represents numbers of photons detected from filtered emission beam 454.

FIG. 2B is a perspective view of a simplified representation of the scanning arm portion of scanner optics and detectors 401. Arm 500 moves in arcs around axis 510, which is perpendicular to the plane of galvo rotation 449. A position transducer 515 is associated with galvanometer 515 that, in the illustrated implementation, moves arm 500 in bi-directional arcs. Transducer 515, in accordance with any of a variety of known techniques, provides an electrical signal indicative of the radial position of arm 500. Certain non-limiting implementations of position transducers for galvanometer-driven scanners are described in U.S. Pat. No. 6,218,803, which is hereby incorporated by reference in its entirety for all purposes. The signal from transducer 515 is provided in the illustrated implementation to a user computer so that clock pulses may be provided for digital sampling of emission signal 492 when arm 500 is in certain positions along its scanning arc, as is described in detail in PCT Application PCT/US01/26297, which is incorporated by reference.

Arm 500 is shown in alternative positions 500′ and 500″ as it moves back and forth in scanning arcs about axis 510. Excitation beams 435 pass through objective lens 445 on the end of arm 500 and excite fluorophore labels on targets hybridized to certain of probes 370 in arrays 132 disposed on slide 333, as described above. The arcuate path of excitation beams 435 is schematically shown for illustrative purposes as path 550. Emission beams 452 pass up through objective lens 445 as noted above. Slide 333 of this example is disposed on translation stage 542 that is moved in what is referred to herein as the “y” direction 544 so that arcuate path 550 repeatedly crosses the plane of arrays 132.

FIG. 2C is a top planar view of arm 500 with objective lens 445 scanning arrays 132 as translation stage 542 is moved under path 550. As shown in FIG. 5B, arcuate path 550 of this example is such that arm 500 has a radial displacement of θ in each direction from an axis parallel to direction 544. What is referred to herein as the “x” direction, perpendicular to y-direction 544, is shown in FIG. 2C as direction 543. Further details of confocal, galvanometer-driven, arcuate, laser scanning instruments suitable for detecting fluorescent emissions are provided in PCT Application PCT/US99/06097 (published as WO99/47964) and in U.S. Pat. Nos. 6,185,030 and 6,201,639, all of which have been incorporated by reference above. It will be understood that although a galvanometer-driven, arcuate, scanner is described in this illustrative implementation, many other designs are possible, such as the voice-coil-driven scanner described in U.S. patent application, Ser. No. 09/383,986, hereby incorporated herein by reference in its entirety for all purposes.

Referring again to FIG. 3, epi-optically active substrate 26 includes a unique array of micro-optical elements 84 with optical properties designed to increase the number of photons interacting with biological material 82 and increase the number of photons collected by lens 24 after interaction with biological material 82. As shown in FIG. 3, micro-optical elements 84 may be formed by microelement structures 86, coated with thin, highly reflective layer 87, and an optical layer 88 having a refractive index greater than the refractive index of surface layers 82 or 90. Optical layer 88 has a surface 92, which includes a surface chemistry binding layer or a ligand 90 arranged to immobilize biological material 82. Reflective layer 87 is made of, for example, a single metal layer, such as gold or aluminum, or several metal layers such, as chromium and gold. Reflective layer 87 is relatively thin, having less than 1000 Å, and preferably less than 200 Å. Optical layer 88 may be created by vapor deposition, sputtering, chemical deposition, or electrochemical deposition. Various methods can be used to form a substantially planar surface 92.

Microelement structures 86 may be formed by microgrooves (having, for example, a triangular cross-section) or cavities having micro-cylindrical, micro-spherical shape, or micro conical section formed, for example, by ellipsoids, paraboloids or hyperboloids. (Alternatively, the microgrooves may have a partially triangular cross-section and partially cylindrical or similar cross-section.) In general, microelement structures 86 and reflecting coating 87 have a shape and surface conditions selected for directing light to a surface 90 at critical angle such that an evanescence wave is created and possibly sustained in resonant mode such as to couple with fluorescent labels attached to biological material 82.

Referring still to FIG. 3, microelement structures 86 include triangularly shaped features designed to reflect light beam 25 toward surface 92 to that most of the reflected light arrives at surface 92 generally at the critical angle. In this embodiment, the triangular features are designed to have approximately symmetrical sides 86 (i.e., approximately the same angle and length). Surface 92 forms a boundary between a material with a high index of refraction (n₂) and another material with a lower index (n₁). At the critical angle θc (sin θc=n₂/n₁) total internal reflection occurs, which excites evanescent field across the interface, having most of the energy contained within less than the wavelength of light. The penetration of the excited evanescent field depends on the interface and the incident angle of the reflected radiation. The evanescent field interacts with biological material 82 located close to the interface. As shown in FIG. 3A, for example, if biological material 82 includes fluorophores, the evanescent field excites fluorescent radiation that is collected by lens 24.

In short, light beam 25 first interacts with semi transparent biological layer 82, and enters high index region 88 at near normal incidence, with little diffraction before arriving at reflective layer 87. Reflective layer 87 is arranged to direct the reflected light generally at an angle to launch evanescent wave 83. Microelements 84 are shaped to reflect light beam 25 away from its incident path and such that preferably, only fluorescent emission from biological material 82 shall be directed toward lens 24. Furthermore, triangularly shaped features 84 (shown in FIG. 3) are achromatic in their ability to achieve total internal reflection.

FIG. 3B illustrates another embodiment of the epi-optically active substrate. Epi-optically active substrate 26A includes an array of micro-optical elements 84A having non-symmetrical shapes designed to generate the evanescent field (at surface 92) more optimally for two or more wavelengths. Similarly as optically active substrate 26, optically active substrate 26A includes micro-optical elements 84A formed by microelement structures and optical layer 88 having a high refractive index. Optical layer 88 has surface 92, which includes binding layer 90 arranged to immobilize biological material 82, as shown in FIG. 3A.

Micro-optical elements 84A includes non-symmetrically shaped triangular features designed to have side 86A shorter than side 86B, i.e., the two sides have different angles relative to the incoming radiation. Reflective layer 87 includes reflective parts 87A and 87B (deposited on respective sides 86A and 86B) designed or optimized to reflect two different wavelengths to achieve total internal reflection and form evanescent field at surface 92.

According to yet another embodiment, epi-optically active substrate 26A includes an array of micro-optical elements having a distribution of not completely symmetrical shapes having somewhat different angles designed to generate the evanescent field at surface 92 for a range of irradiation wavelengths. This distribution of shapes may also be somewhat random due to the fabrication process.

FIG. 4 illustrates another embodiment of an epi-optically active substrate. Epi-optically active substrate 26B includes an array of micro-optical elements 100 having cylindrical or spherical shapes distributed over a region or layer 102. Micro-optical elements 100 include a reflective layer 105 designed to reflect irradiation light toward surface 104 and fluorescent light toward lens 24. Similarly as optically active substrate 26, optically active substrate 26B includes micro-optical elements 100 formed by microelement cavities 106 and an optical layer 108 having a high refractive index. Optical layer 108 has surface 104, which includes binding layer 110 arranged to immobilize biological material. In this embodiment, the biological material is located on the proximal (near) side with respect to the irradiation beam 25.

Reflective layer 105 is relatively thin, having less than 1000 Å, and preferably less than 200 Å. Reflective layer 105 is made of, for example, a single metal layer, such as gold or aluminum, or several metal layers such, as chromium and gold. Optical layer 108 may be created by vapor deposition, sputtering, chemical deposition, or electrochemical deposition. The shape of micro-element 100 and shape of outer surface 104 of high index coating 108 cooperate to capture and redirect fluorescence emission from the biological material (as shown in FIG. 4A) such that it may be captured in totality or in part by lens 24 and transmitted to detector 36 (or detector 70). Optical features 106 can be manufactured in support 101 by embossing, injection molding, photolithography, or other conventional means. Optical layer 108 is made of a high index material such as TiO₂ or TeO₂ or others.

The size and period of micro-optical elements 100 are preferably such that micro-optical elements 100 within the area illuminated at any defined pixel by beam 25 do not cause excessive artifact, wherein the illuminated area has a radius of about 2 μm to about 10 μm. The radius of the having cylindrical or spherical features 100 is in the range of about 2,000 Å to about 5,000 Å, and optical layer 104 is located at least about 100 Å above the features. Therefore, the period is between about 0.2 μm and about 5.0 μm, and preferably between about 0.35 μm and about 2.0 μm. This may cause diffraction effect as well as scatter effects to induce surface wave creation. The periodic distance between micro elements or micro structure preferably enhances diffraction effects that launches light incident onto it.

Referring to FIGS. 4B and 4C, because the diffraction effect is correlated to the wavelength and angle of incident light, grooves or optical features may incorporate a suitable selection of distances so as to accommodate a selected number of wavelengths. Instead of spherical cavities, micro-optical elements 100 may be formed by parallel or semi-parallel half cylinders, parallel or semi-parallel hyperbolic structures, or other similar microstructures. FIG. 4B illustrates a top view of micro-optical elements 100 formed by semi-parallel half cylindrical features having variable spacing. FIG. 4C illustrates a top view of micro-optical elements 100 formed by of spherical cavities. Furthermore, instead of spherical cavities, micro-optical elements 100 may be formed to have conical shapes, or any conic sections such as formed by ellipses, paraboloids or hyperboloids.

Referring again to FIG. 3, micro-optical elements 84 may be formed by microlens structures 86, such as spherical cavities with a radius in the range of 100 nm to 100 μm. The diameter and depth of the spherical microlens cavities defines the thickness of the high index layer 88. Layer 88 may partially or completely fill the cavities. High index coating 88 includes titanium dioxide with an index of refraction of 2.4, gallium phosphate with an index of refraction 3.4, or other medium with suitable index of refraction. High index layer 88 is designed to have a thickness depending on its transmission coefficient so that a relatively low transmission coefficient will cause acceptable optical losses.

The microstructures can again be created in the substrate by forced embossing at a proper temperature, or by casting against a suitably formed negative master (as used when creating CD and DVD discs) or combination of both. As described above, the substrate may include a PMMA®, Plexiglas® or a similar plastic with an index of refraction of about 1.57. The high index coating may include titanium dioxide, Tellurium dioxide, gallium phosphate or other medium with suitable index of refraction in the range of 2 to 4 and preferably an index of refraction between about 2 and 3.5.

According to one embodiment, optical plate 80 (or 101) is made of an optically transparent material such as PMMA®, Plexiglas® or a similar plastic. Microelements 84 (or 100) can be created in plate 80 (or 101) by forced embossing at a proper temperature, or by casting the plate against a suitably formed negative master or a combination of both (as used when creating CD and DVD discs). Alternatively, plate 80 and micro-optical elements 84 could be made of etched glass, quartz or another material. Alternatively, plate 80 includes an optically opaque material such as aluminum or brass and micro-optical elements may be diamond machined on air bearing lathe or milling machines such as Pneumo Precision machines.

Referring still to FIG. 4, after irradiation, optically active substrate 26B receives, at a surface 110, the excitation light over light path 25. The excitation light first illuminates near transparent thin (approximately 20 to 200 Angstroms) biological material and is diffracted inside optically transparent high index layer 108. The dense array of micro-optical elements 110 includes reflective coating 105 over all micro-features 106. Reflective coating 105 reflects and concentrates the transmitted photons by increasing the number of photons interacting with fluorophores 90 in the biological material. Furthermore, the dense array of micro-optical elements 100 increases the number of photons of the excited radiation transmitted back to lens 24, and thus the number of photons collected by lens 24. Therefore, the dense array of micro-optical elements 100 enables an increase in the signal-to-noise ratio.

Referring to FIG. 1, optical system 10 collects the reflected and fluorescent light by lens 24 and delivers this light over light path 30 and light path 33 to detector 36. Lens 32 receives fluorescent light transmitted through dichroic mirror 20 over a light path 30 and focuses the light onto pinhole 34 and possibly rejection or band pass filter 31. Detector 36 detects fluorescent light emitted from a depth of pixel A corresponding to the location of the examined biological material on the proximal surface of optically active substrate 26B.

In general, a probe is a surface-immobilized molecule that is recognized by a particular target. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

A target is a molecule that has an affinity for a given probe. Targets may be naturally-occurring or manmade molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “probe target pair” is formed when two macromolecules have combined through molecular recognition to form a complex.

Generally, the sample nucleic acid for which sequence information is desired is contacted with the array. This “target” sequence is typically labeled with a detectable group such as a fluorescent moiety, i.e., fluorescein or rhodamine. Following hybridization of the target to the array, one can detect the position on the array to which the target sequence binds by scanning the surface of the array for fluorescence.

The surface is typically scanned by directing excitation radiation at the surface to activate the fluorescent labeling group in the target, which in turn emits a fluorescent response radiation. The fluorescent response radiation is detected and assigned to the region from which it originated. By knowing the position from which the fluorescence originates, one can identify the sequence to which the target binds.

Although generally used herein to define separate regions containing differing polymer sequences, the term “feature” generally refers to any element, e.g., region, structure or the like, on the surface of a substrate. Typically, substrates to be scanned using the scanning systems described herein, will have small feature sizes, and consequently, high feature densities on substrate surfaces. For example, individual features will typically have at least one of a length or width dimension that is no greater than 100 μm, and preferably, no greater than 50 μm, and more preferably no greater than about 20 μm. Thus, for embodiments employing substrates having a plurality of polymer sequences on their surfaces, each different polymer sequence will typically be substantially contained within a single feature.

The probe arrays will have a wide range of applications. For example, the probe arrays may be designed specifically to detect genetic diseases, either from acquired or inherited mutations in an individual DNA. These include genetic diseases such as cystic fibrosis, diabetes, and muscular dystrophy, as well as acquired diseases such as cancer.

Genetic mutations may be detected by a method known as sequencing by hybridization. In sequencing by hybridization, a solution containing one or more targets to be sequenced (i.e., samples from patients) contacts the probe array. The targets will bind or hybridize with complementary probe sequences. Generally, the targets are labeled with a fluorescent marker, radioactive isotopes, enzymes, or other types of markers. Accordingly, locations at which targets hybridize with complimentary probes can be identified by locating the markers. Based on the locations where hybridization occurs, information regarding the target sequences can be extracted. The existence of a mutation may be determined by comparing the target sequence with the wild type.

Substrate 26 may be fabricated of, or may include, a wide range of material, either biological, non-biological, organic, inorganic, or a combination of any of these, existing as particles, strands, precipitates, gels, sheets, tubing, spheres, containers, capillaries, pads, slices, films, plates, slides, etc. The substrate may have any convenient shape, such as a disc, square, sphere, circle, etc. The substrate is preferably flat but may take on a variety of alternative surface configurations. For example, the substrate may contain raised or depressed regions on which a sample is located. The substrate and its surface preferably form a rigid support on which the sample can be formed. The substrate and its surface are also chosen to provide appropriate light-absorbing characteristics. For instance, the substrate may be a polymerized Langmuir Blodgett film, functionalized glass, Si, Ge, GaAs, GaP, SiO₂, SiN₄, modified silicon, or any one of a wide variety of gels-or polymers such as (poly)tetrafluoroethylene, (poly)vinylidenedifluoride, polystyrene, polycarbonate, or combinations thereof. Other materials with which the substrate can be composed of will be readily apparent to those skilled in the art upon review of this disclosure.

While the invention has been described with reference to the above embodiments, the present invention is by no means limited to the particular constructions described and/or shown in the drawings. The present invention also comprises any modifications or equivalents within the scope of the following claims.

1. An optical apparatus for examination of biological material, comprising:
an optical substrate having a surface comprising a dense array of micro-optical elements in close proximity to biological material being examined; and
a light source, a light detector, and optical elements for providing light from said light source to said biological material and from said biological material to said light detector, said optical elements including a lens arranged to provide light to and collect light from said dense array of micro-optical elements and said biological material, wherein said lens and said dense array of micro-optical elements are constructed to have optical properties that increase the number of photons interacting with said biological material and increase the number of photons collected by said lens after interaction with said biological material.

2. The optical apparatus of claim 1 wherein said substrate includes a reflective layer opaque to said light emitted from said light source.

3. The optical apparatus of claim 1 wherein said lens is a final objective lens.

4. The optical apparatus of claim 3 wherein said final objective lens is positioned in proximity of said optical substrate.

5. The optical apparatus of claim 3 wherein said objective lens includes a flying objective lens mounted on a scanning apparatus.

6. The optical apparatus of claim 3 or 4 wherein said objective lens weighs less than about 2 grams.

7. The optical apparatus of claim 3 or 4 wherein said objective lens has a numerical aperture larger than about 0.1.

8. The optical apparatus of claim 1 wherein said optical elements include a pinhole constructed and arranged for confocal imaging.

9. The optical apparatus of claim 5 wherein said light source includes a laser source

10. The optical apparatus of claim 1 wherein said light source includes a one-dimensional array of source elements co-operatively arranged with said optical elements to deliver collimated light to said dense array of micro-optical elements, and said light detector includes a one-dimensional array of detector elements.

11. The optical apparatus of claim 1 wherein said light source includes a two-dimensional array of source elements co-operatively arranged with said optical elements to deliver collimated light to said dense array of micro-optical elements, and said light detector includes a two-dimensional array of detector elements.

12. The optical apparatus of claim 1 wherein said optical substrate includes an array of features located in proximity of said dense array of micro-optical elements.

13. The optical apparatus of claim 12 wherein each of said features includes several million DNA molecules per feature.

14. The optical apparatus of claim 12 wherein said array of features includes oligonucleotide microarrays to which fluorescently labeled DNAs or RNAs are bound.

15. The optical apparatus of claim 1 wherein said optical substrate includes polypeptides or other polymer arrays located at said dense array of micro-optical elements.

16. The optical apparatus of claim 3 further including an oscillating support structure constructed to carry and periodically scan said objective lens over said substrate.

17. The optical apparatus of claim 16 wherein said oscillating support structure includes a rectilinear scanning module carrying said objective lens.

18. The optical apparatus of claim 17 wherein said oscillating support structure includes a rotational scanning module carrying said objective lens.

19. The optical apparatus of claim 1 wherein at least one of said micro-optical elements has a dimension comparable to the wavelength of light emitted from said light source.

20. The optical apparatus of claim 1 wherein said micro-optical elements include micro-lenses.

21. The optical apparatus of claim 18 wherein the micro-lenses are formed by micro-cavities formed inside the substrate.

22. The optical apparatus of claim 18 wherein the micro-lenses are formed by micro-cavities having parallel or semi-parallel groves in the form of half cylinders or quarter cylinders.

23. The optical apparatus of claim 21 wherein the micro-cavities are formed inside the substrate by spherical indentations about one radius in depth.

24. The optical apparatus of claim 21 wherein the micro-cavities are formed inside the substrate by spherical indentations having a depth less than a radius of the sphere.

25. The optical apparatus of claim 21 wherein the micro-cavities are formed inside the substrate by indentations having a hyperbolic shape.

26. The optical apparatus of claim 21 wherein the micro-cavities include a surface covered by a layer of a high index medium transparent at a wavelength of said light.

27. The optical apparatus of claim 26 wherein the high index medium substantially fills said micro-cavities.

28. The optical apparatus of claim 1 wherein the micro-optical elements are micro-lenses formed by micro-cavities inside the substrate, the micro-cavities having a radius in the range of 0.1 micron μm to 10 micron μm.

29. The optical apparatus of claim 1 wherein the micro-optical elements are micro-lenses formed by micro-cavities inside the substrate, the micro-cavities having a radius less than 100 micron (μm).

30. The optical apparatus of claim 1 wherein the micro-optical elements are micro-lenses formed by micro-cavities inside the substrate and wherein a diameter and depth of the micro-cavities define the thickness of a high index coating deposited on the surface.

31. The optical apparatus of claim 1 wherein the substrate is made of a material transparent to fluorescent light emitted from fluorophores excited at their emission wavelength.

32. The optical apparatus of claim 26 wherein the high index coating has a thickness of about 10 angstrom to 1000 angstrom depending on the material so that a relatively low coefficient of transmission of the material causes acceptable optical losses.

33. The optical apparatus of claim 26 wherein the high index coating includes titanium dioxide with an index of refraction of about 2.4.

34. The optical apparatus of claim 26 wherein the high index coating includes gallium phosphate with an index of refraction of about 3.4.

35. An optical apparatus for examination of biological material, comprising:
an optical substrate having a first surface and a second surface opposite to the first surface, the first surface comprising a dense array of micro-optical elements in close proximity to biological material being examined; and
a light source, a light detector, and optical elements for providing light from said light source to said biological material and from said biological material to said light detector, said dense array of micro-optical elements is constructed to increase the number of photons interacting with said biological material and increase the number of photons detected by said light detector after interaction with said biological material.

36. The optical apparatus of claim 35 wherein the micro-optical elements include micro-structures formed at said first surface.

37. The optical apparatus of claim 36 wherein said micro-structures include micro-lenses.

38. The optical apparatus of claim 35 wherein the micro-optical elements include micro-structures formed inside said substrate.

39. The optical apparatus of claim 38 wherein said micro-structures formed inside said substrate include micro-lenses.

40. The optical apparatus of claim 39 wherein said micro-lenses include micro-cavities inside said substrate, and wherein a diameter and depth of the micro-cavities define the thickness of a high index coating deposited on the first surface.

41. An optically active substrate for use with an optical system constructed for examination of biological materials, comprising:
an optical substrate having a first surface and a second surface opposite to the first surface; and
a dense array of micro-optical elements associated with said optical substrate and located in close proximity to biological material being examined, said dense array of micro-optical elements being constructed to have optical properties that increase the number of irradiation photons interacting with said biological material and increase the number of photons provided to an external optical system after interaction of said irradiation photons with said biological material.

42. The optical apparatus of claim 41 wherein the micro-optical elements include micro-structures formed at said first surface.

43. The optical apparatus of claim 42 wherein said micro-structures include micro-lenses.

44. The optical apparatus of claim 41 wherein the micro-optical elements include micro-structures formed inside said substrate.

45. The optical apparatus of claim 44 wherein said micro-structures formed inside said substrate include micro-lenses.

46. The optical apparatus of claim 45 wherein said micro-lenses include micro-cavities inside said substrate, and wherein a diameter and depth of the micro-cavities define the thickness of a high index coating deposited on the first surface.

47. A method of examination of biological material, comprising the act of:
providing an optically active substrate of claim 41;
irradiating said first surface with optical radiation of a selected wavelength; and
detecting photons that have interacted with said biological material.

48. The method of claim 47 further including creating an image using said detected photons.
